(19) United States
(12) Patent Application Publication
(10) Pub. No.: US 2018/0147348 A1
(43) Pub. Date: May 31, 2018

(54) AUDIO BOLUS WITH IMPROVED SAFETY FOR USE IN A DRUG DELIVERY SYSTEM

(71) Applicant: Animas Corporation, West Chester, PA (US)

(72) Inventors: Thomas MCCANN, Pottstown, PA (US); Daniel FINAN, Philadelphia, PA (US); Anthony DIUBALDI, Wayne, PA (US); Brian LEVY, New York, NY (US)

(21) Appl. No.: 15/822,529

(22) Filed: Nov. 27, 2017

Related U.S. Application Data
(60) Provisional application No. 62/427,965, filed on Nov. 30, 2016.

(51) Int. Cl. A61M 5/172

(52) U.S. Cl. CPC A61M 5/1723; A61M 2205/581; A61M 2230/201; A61M 2205/18; A61M 2205/52; A61M 2205/3584; A61M 2205/50; A61M 2205/8206; A61M 2205/502

(57) ABSTRACT

The invention provides for disabling of the audio bolus feature of a drug delivery system on the occurrence, or predicted occurrence, of certain events mitigating the problem of a user delivering unneeded drug. The invention provides a drug delivery system having at least one controller configured to autonomously modulate drug delivery from the drug delivery device, a sensor for measuring an analyte of the user, and an audio bolus feature, wherein the audio bolus feature is automatically disabled if the controller determines a therapeutically significant state is present.

### CROSS REFERENCE TO RELATED APPLICATION

This application claims priority under relevant portions of 35 U.S.C. § 119 to United States Application No. 62/427,965 filed, Nov. 30, 2016, the entire contents of which are hereby incorporated by reference.

### FIELD OF THE INVENTION

This invention relates to the use of audio-bolusing in a drug delivery system. More specifically, the invention is directed to an audio bolus with improved safety for use in a system, such as a closed loop drug delivery system, artificial pancreas, or the like, which system is controlled by an autonomous drug delivery modulating algorithm.

### BACKGROUND OF THE INVENTION

Diabetes mellitus is a chronic metabolic disorder caused by an inability of the pancreas to produce sufficient amounts of the hormone insulin, resulting in the decreased ability of the body to metabolize glucose. This failure leads to hyperglycemia, or the presence of an excessive amount of glucose in the blood plasma. Persistent hyperglycemia alone or in combination with hypoinsulinemia is associated with a variety of serious symptoms and life threatening long term complications. Because restoration of endogenous insulin production is not yet possible, a permanent therapy is necessary that provides constant glycemic control in order to always maintain the level of blood glucose within normal limits. Such glycemic control is achieved by regularly supplying external insulin to the body of the patient to reduce elevated levels of blood glucose.

Substantial improvements in diabetes therapy have been achieved by the development of drug delivery devices that relieve the patient of the need to use syringes or drug pens for the administration of multiple daily injections. These drug delivery devices allow for the delivery of drug in a manner that bears greater similarity to the naturally occurring physiological processes and can be controlled to follow standard or individually modified protocols to give the patient better glycemic control.

These drug delivery devices can be constructed as implantable devices. Alternatively, the device may be an external device with an infusion set for subcutaneous infusion to the patient via the transcutaneous insertion of a catheter, cannula, or transdermal drug transport such as through a patch. The external drug delivery devices are mounted on the body of clothing or, and more preferably, hidden beneath or inside clothing, and are generally controlled via a user interface built-in to the device or provided on a separate remote device.

Blood or interstitial glucose monitoring is required to achieve acceptable glycemic control with the devices. For example, delivery of suitable amounts of insulin by the drug delivery device requires that the user frequently, episodically determines his or her blood glucose level by testing the blood glucose level. The level value is manually input into a user interface for the external pumps or controller, after which a suitable modification may be calculated to the default or currently in-use insulin delivery protocol, i.e., dosage and timing, which modification is used to adjust the drug delivery device operation accordingly. Alternatively, or in conjunction with episodic determination, continuous glucose monitoring (“CGM”) may be used with drug delivery devices, which CGM allows for closed loop control of the insulin being infused into the diabetic patient.

To allow for closed-loop control, autonomous modulation of drug being delivered to the user is provided by a controller using one or more control algorithms. For example, proportional-integral-derivative (“PID”) controllers that are reactive to observed glucose levels may be utilized, which controllers can be tuned based on simple rules of the mathematical models of the metabolic interactions between glucose and insulin in a person. Alternatively, a model predictive controller (“MPC”) may be used. The MPC is advantageous because the MPC pro-actively considers the near future effects of control changes and constraints in determining the output of the MPC, whereas PID typically involves only past outputs in determining future changes. Constraints can be implemented in the MPC such that the MPC prevents the system from exceeding a limit that has been reached. Another benefit of MPC use is that the model in the MPC can, in some cases, theoretically compensate for dynamic system changes whereas in a feedback control, such as PID control, such dynamic compensation would not be possible. Known MPCs are described in the following documents: U.S. Pat. No. 7,060,059; U.S. Patent Publication Nos. 2011/0313680 and 2011/0257627; International Publication WO 2012/051344; Percival et al., “Closed-Loop Control and Advisory Mode Evaluation of an Artificial Pancreatic β Cell: Use of Proportional-Integral-Derivative Equivalent Model-Based Controllers” J. Diabetes Sci. Technol., Vol. 2, Issue 4, July 2008; Paola Soru et al., “MPC Based Artificial Pancreas; Strategies for Individualization and Meal Compensation” Annual Reviews in Control 36, p. 118-128 (2012); Cobelli et al., “Artificial Pancreas: Past, Present, Future” Diabetes, Vol. 60, November 2011; Magni et al., “Run-to-Run Tuning of Model Predictive Control for Type 1 Diabetes Subjects: In Silico Trial” J. Diabetes Sci. Technol., Vol. 3, Issue 5, September 2009; Lee et al., “A Closed-Loop Artificial Pancreas Using Model Predictive Control and a Sliding Meal Size Estimator” J. Diabetes Sci. Technol., Vol. 3, Issue 5, September 2009; Lee et al., “A Closed-Loop Artificial Pancreas based on MPC: Human Friendly Identification and Automatic Meal Disturbance Rejection” Proceedings of the 17th World Congress, The International Federation of Automatic Control, Seoul Korea Jul. 6-11, 2008; Magni et al., “Model Predictive Control of Type 1 Diabetes: An in Silico Trial” J. Diabetes Sci. Technol., Vol. 1, Issue 6, November 2007; Wang et al., “Automatic Bolus and Adaptive Basal Algorithm for the Artificial Pancreatic β-Cell” Diabetes Technol. Ther., Vol. 12, No. 11, 2010; Percival et al., “Closed-Loop Control of an Artificial Pancreatic β-Cell Using Multi-Parametric Model Predictive Control” Diabetes Res. 2008; Kovatchev et al., “Control to Range for Diabetes: Functionality and Modular Architecture,” J. Diabetes Sci. and Technol., Vol. 3, Issue 5, September 2009; and Atlas et al, “MD-Logic Artificial Pancreas System,” Diabetes Care, Vol. 33, No. 5, May 2010. All articles or documents cited in this application are hereby incorporated by reference in their entireties.

In addition to autonomous modulation of basal drug delivery, some of the systems for drug delivery provide for bolus amounts of drug to be delivered via the use of audio-bolusing. The audio bolus feature is desirable because the user may facilitate drug delivery more discreetly without the need to look at the delivery device.

However, the audio bolus feature also may be disadvantageous in that there may be information residing on the delivery device or another device of the system that, if known by the user, may contradict the user's perception of a need for a bolus. Such information may be accessible only if the user looks at the device, or another device in the system, such as a remote controller, smart-phone or other device that is capable of receiving information about the user's glucose status. Therefore, incorporating a feature into the system that disables the user's audio bolus capability in certain circumstances is desirable.

### BRIEF DESCRIPTION OF THE DRAWINGS

FIG. 1 depicts an embodiment of an insulin delivery system useful with the invention.

FIG. 2 depicts another embodiment of an insulin delivery system useful with the invention.

FIG. 3 depicts a schematic of the internal hardware architecture of a drug delivery device useful with the invention.

FIG. 4 is a flow diagram of a process in accordance with an embodiment of the invention.

### DETAILED DESCRIPTION OF THE INVENTION

As used herein, the term “user” refers to any human or animal subject and is not intended to limit the systems or methods to human use, although use of the subject invention in a human patient represents a preferred embodiment. Furthermore, the term “user” includes not only the human or animal using a drug delivery system but also the caretakers (e.g., parent or guardian, nursing staff, home care employee and the like). The term “drug” refers to a hormone, biologically active materials, pharmaceuticals or other chemicals that cause a biological response (e.g., glycemic response) in the body of a user or patient and, preferably, is insulin.

It is a discovery of the invention that by providing for disabling of the audio bolus feature of a drug delivery system on the occurrence, or predicted occurrence, of certain events, the problem of a user delivering unneeded drug may be avoided. The invention provides a drug delivery system comprising, consisting essentially of and consisting of: a drug delivery device; at least one controller configured to autonomously modulate drug delivery from the drug delivery device; a sensor for measuring an analyte of the user; and an audio bolus feature, wherein the audio bolus feature is automatically disabled if the controller determines a therapeutically significant state is present.

For purposes of this invention, by “audio bolus feature” is meant an element of a drug delivery system in which each increment of a drug, preferably insulin, bolus to be delivered that is entered into the system, as well as the total amount entered, and optionally the delivery itself, is confirmed via an audible sound issued by a device of the system alleviating the need for the user to look at any device of the system to initiate and administer the bolus. The sound may be any audible tone, beep, note, speech or the like that is suitable for audibly alerting the user. The audio bolus increments and delivery command may be entered via use of a physical input key, button or the like, touch screen or voice input to one or more of the drug delivery device or any other device in the system that is used to control the drug delivery device.

Additionally for purposes of the invention, by “therapeutically significant state” is meant that, as determined by one or more components of the system, the system user presently exhibits, or is predicted to exhibit, an undesirable physiological state. In a preferred embodiment, the therapeutically significant state is exacerbated or caused by either or both (1) the autonomous infusion by the system's control algorithm of additional drug above the basal amount delivered and (2) delivery of a bolus of drug. In the case in which the drug delivery system is delivering a drug for management of blood glucose levels, such as insulin, the therapeutically significant state is hypoglycemia or a predicted, impending occurrence of hypoglycemia, and the therapeutically significant state may be reflected in the estimated current insulin active in the user's body resulting from delivery of insulin, such current, active insulin commonly known as “insulin on board” or “IOB.” Optionally, the therapeutically significant state may also be based on one or more of a current CGM reading, or an extrapolation or other model prediction of current CGM data into the future which may or may not incorporate IOB, other measures of the effect of insulin on the resulting glucose concentration within the body, or other therapeutic parameters such as a measure of insulin sensitivity.

In a preferred embodiment of the invention, the therapeutically significant state is determined to be present when the current IOB resulting from the insulin delivered by bolus, by the pump autonomously via an automated delivery algorithm, or both is greater or equal to a percentage of the current basal delivery rate multiplied by a selected time period or:

current IOB value≥preselected IOB value

wherein the preselected IOB is determined using the formula

X%*(CBR*T)

wherein X is the percentage of the current basal delivery rate;
“CBR” is the then current basal delivery rate in units per hour of the device; and
T is a selected time period.

Thus, for example, if X is selected as 50%, the CBR is 1 unit/h of insulin, and T is 1 hour, then:

50*(1U/h*1h)

would equal 0.5 units. If a current value for IOB, or Y, is 0.5 units, because that value is equal to or greater than the pre-selected 0.5 units, when the audio bolus command is initiated by the device user, then the audio bolus will be disabled by a component within the system because a therapeutically significant state has been determined to exist. The user will receive one or both of an auditory or vibratory alarm to indicate the audio bolus is disabled. Additionally, the device display on the pump, and optionally or alternatively the display of another device within the delivery system, will display to the user notice of the disablement, and preferably also the IOB calculated. The disablement will recur upon any future initiation of the audio bolus command by the device user as long as Y, the current IOB, is greater than or equal to the preselected value, such as the exemplified 0.5 units.

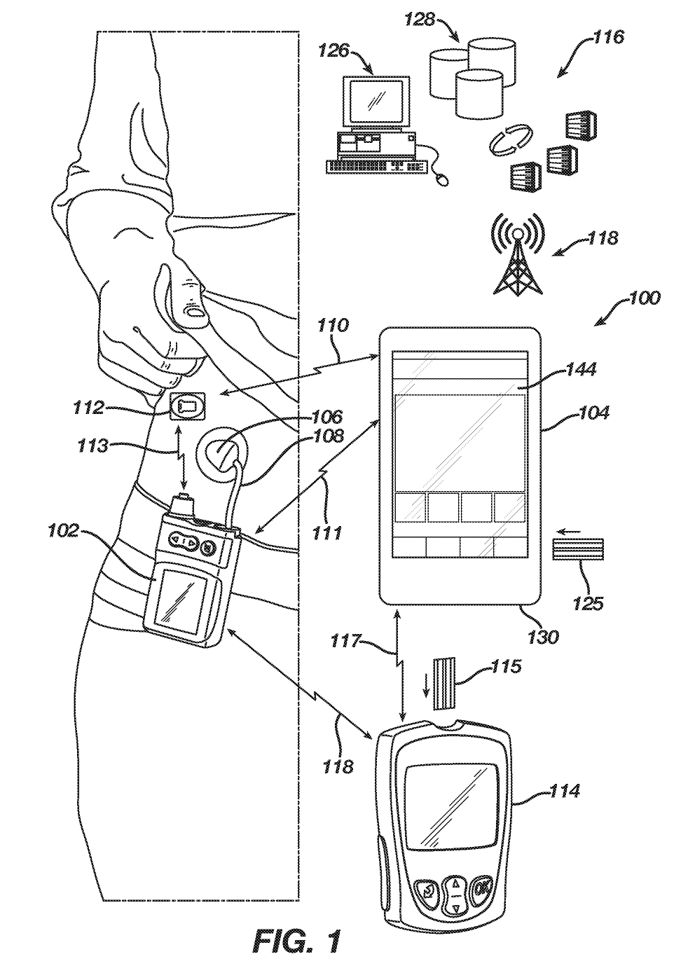

A preferred drug delivery system 100 of the invention in which the invention may be used is illustrated in FIG. 1. Drug delivery device 102 and a controller 104 are shown, which controller is capable of at least some operational control of the device. Drug delivery device 102 as shown is connected to an infusion set 106 via flexible tubing 108.

Drug delivery device 102 may be, and preferably is, configured to transmit and receive data to and from controller 104 by, for example, radio frequency communication (“RF”), BLUETOOTH® communication, including BLUETOOTH® low energy communication, or the like. Also, it is configured to wirelessly receive glucose data from a CGM sensor 112. Alternatively, drug delivery device 102 may function as a stand-alone device.

In one embodiment, drug delivery device 102 is an insulin infusion pump and controller 104 is a hand-held portable, controller. The controller may take any of a number of forms. For example, the controller may be a device the sole purpose of which is to control the drug delivery device. Alternatively, the controller may form a part of another device as for example, an episodic blood glucose meter or a consumer electronic device such as a smart-phone, exercise monitoring device, personal digital assistant and the like. In those embodiments in which the controller forms a part of another device, the controller may be combined in the device into either an integrated, monolithic device or separable devices that are attachable to each other, as for example by docking, to form an integrated device. Each of the controller embodiments includes a suitable microcontroller (not shown) that is programmed to carry out various functions.

In any such embodiment using a controller, data transmitted from the drug delivery device to the controller may include any information collected by the delivery device such as, for example, blood glucose information, IOB, insulin delivery data, basal dosing, bolus dosing, insulin to carbohydrates ratio, insulin sensitivity factor, and the like. Alternatively, the glucose data from the continuous glucose sensor 112 may be transmitted directly to the controller 104 through a wireless communication channel 110. Data also may be transmitted from remote controller 104 or blood glucose meter 114 through a wireless communication channel 110 and 188, respectively. Data also may be transmitted between remote controller 104 and insulin delivery device 102 and may include any of a wide variety of data such as episodic glucose test results, a food database to allow the drug delivery device 102 to calculate the amount of insulin to be delivered, basal dosing, bolus calculating, bolus dosing and the like. In yet another alternative embodiment, and as shown in FIG. 1, an episodic blood glucose meter 114 may be used alone or in conjunction with the CGM sensor 112 to provide data to either or both of the controller 104 and drug delivery device 102.

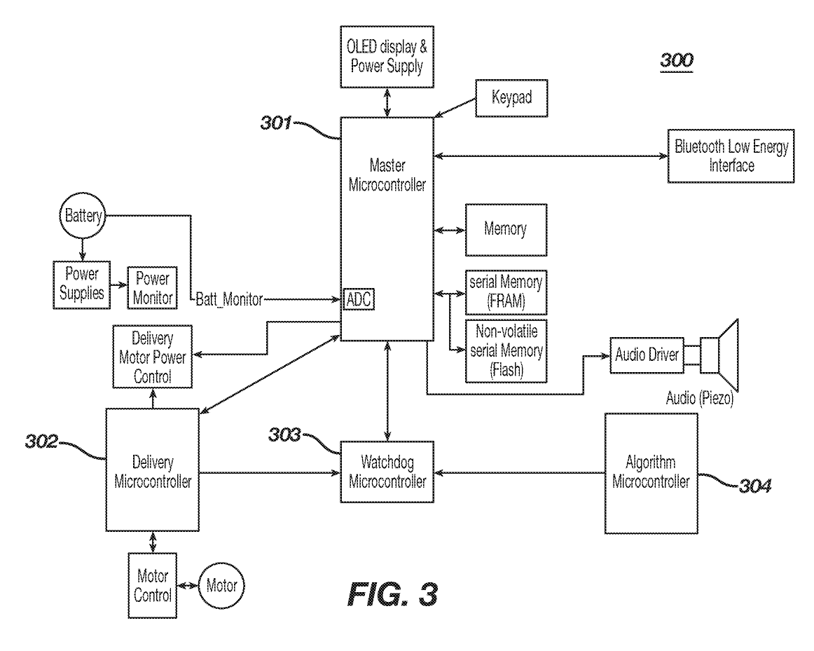

In one embodiment, the drug delivery device is configured to include at least one microcontroller configured to implement one or more autonomous control algorithms that use received continuous glucose readings from the CGM sensor to autonomously modulate drug delivery. In another embodiment, the autonomous control algorithm may be contained in a controller that is a device within the system and other than the drug delivery device. In a more preferred embodiment, the drug delivery device incorporates multiple microcontrollers. For example, as shown in FIG. 3, master, delivery, watchdog and algorithm microcontrollers (301, 302, 303, and 304, respectively) are incorporated within the drug delivery device 300. In this embodiment, the algorithm microcontroller contains the autonomous control algorithm.

The drug delivery device may also be configured for bi-directional wireless communication with, as shown in FIG. 1, a remote health monitoring station 116 through, for example, a wireless communication network 118. Remote controller 104 and remote monitoring station 116 also may be configured for bi-directional, wired communication through, for example, a telephone land based communication network. Remote monitoring station 116 may be used, for example, to download upgraded software to drug delivery device 102 and to process information from drug delivery device 102. Alternatively, software downloading may be carried out through controller 104. Examples of remote monitoring station 116 may include, but are not limited to, a personal or networked computer 126, server 128 to a memory storage, a personal digital assistant, mobile telephone such as a smart-phone, a hospital-based monitoring station, or a dedicated remote clinical monitoring station. Alternatively and though not shown in the figures, storage, for example, the control algorithm, may further be provided in whole or in part by cloud storage.

Drug delivery devices useful in the invention may include electronic signal processing components including a central processing unit and memory elements for storing control programs and operation data, a radio frequency module, BLUETOOTH interface, or the like for sending and receiving communication signals (i.e., messages) to and from remote controller, a display for providing operational information to the user, at least one and preferably a plurality of navigational buttons for the user to input information, a battery for providing power to the system, alarms (e.g., visual, auditory or tactile) for providing feedback to the user, a vibrator for providing feedback to the user, a drug delivery mechanism (e.g. a drug pump and drive mechanism) for forcing a drug, such as insulin, from a reservoir (e.g., a insulin cartridge) through a side port connected to an infusion set, such as 108/106 shown in FIG. 1, and into the body of the user. An example of a drug delivery device can be in the form of a modified Animas® VIBE™ insulin pump manufactured by Animas Corporation, Wayne, Pa. U.S.A.

User glucose levels or concentrations can be determined by the use of any suitable CGM sensor. For example, the CGM sensor may utilize amperometric electrochemical sensor technology to measure glucose with three electrodes operably connected to the sensor electronics and covered by a sensing membrane and a biointerface membrane, which are attached by a clip. In such a case, the top ends of the electrodes are in contact with an electrolyte phase, which is a free-flowing fluid phase disposed between the sensing membrane and the electrodes. The sensing membrane may include an enzyme, e.g., glucose oxidase, which covers the electrolyte phase. In this exemplary sensor, the counter electrode is provided to balance the current generated by the species being measured at the working electrode. In the case of a glucose oxidase based glucose sensor, the species being measured at the working electrode is H₂O₂. The current that is produced at the working electrode (and flows through the circuitry to the counter electrode) is proportional to the diffusional flux of H₂O₂ generated by this electrochemical transformation of glucose into its enzymatic byproducts. Accordingly, a raw signal may be produced that is representative of the concentration of glucose in the user's body, and therefore may be utilized to estimate a meaningful glucose value. Details of the sensor and associated components are shown and described in U.S. Pat. No. 7,276,029, which is incorporated by reference herein as if fully set forth herein this application. In one embodiment, a continuous glucose sensor such as the Dexcom G4® or G5® (manufactured by Dexcom Inc.) may be utilized with the exemplary embodiments described herein.

In one embodiment of the invention, the following components can also be utilized as a system for management of diabetes that is akin to an artificial pancreas: a drug delivery device (infusion pump); an episodic glucose sensor; a continuous glucose monitoring system with interface to connect these components and programmed in MATLAB® language and accessory hardware to connect the components together; and control algorithms in the form of PID or MPC that automatically and autonomously regulates the rate of insulin delivery based on the glucose level of the patient, historical glucose measurement and, in the case of MPC, anticipated future glucose trends, and patient specific information. Alternatively, a smart phone or other remote device may be used to control the drug delivery device and send and receive information from carious devices within the system.

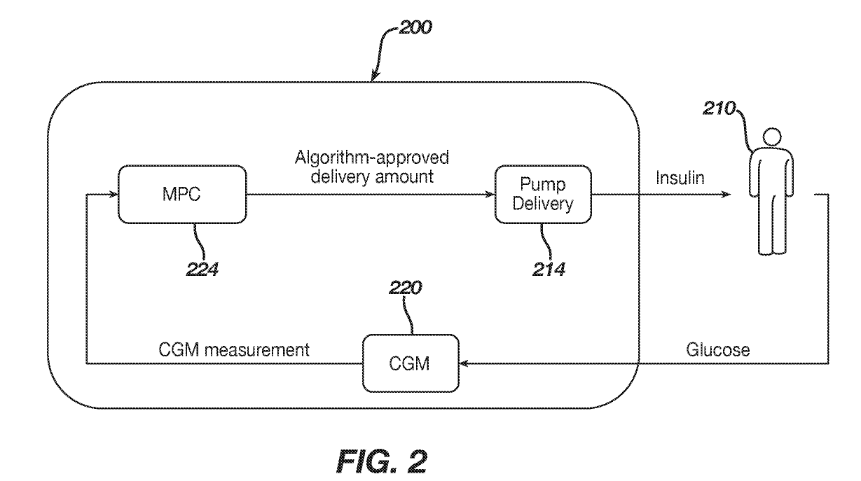

Referring to FIG. 2, there is shown an embodiment of a drug delivery device 200 for use with the invention, shown schematically, for use in conjunction with a patient 210. The drug delivery device 200 according to this embodiment houses a pump delivery module 214, CGM module 220 and MPC module 224. This embodiment, as well as that of other embodiments disclosed herein, preferably employ a hypoglycemia-hyperglycemia minimizer (“HHM”), as for example disclosed in U.S. Pat. No. 8,562,587 and U.S. application Ser. No. 14/015,831 both incorporated in their entireties by reference, more preferably all integrated within the housing or MPC of the drug delivery device. The CGM module is configured for receiving signals from a CGM sensor placed on the patient or user. As shown in FIG. 2, the MPC module 224 is operatively connected to the CGM module 220 as well as the pump delivery module 214 and receives subcutaneous glucose information for providing the same to a stored algorithm, which is also made aware of all previous deliveries of insulin.

As shown in FIG. 3, the MPC (or algorithm) module or microcontroller 304 is operatively connected to the master microcontroller (or module) 301 and delivery microcontroller 302 through watchdog controller 303. Master microcontroller 301 tracks all previous deliveries of insulin. The insulin delivery data is used to calculate near-future predictions of glucose levels and produce an insulin delivery rate that mitigates the near-future predicted, or actual, hyper- or hypoglycemic conditions. The rate is then actuated by the pump delivery module (214 in FIGS. 2 and 302 in FIG. 3) relative to the patient set rate corresponding to the current (e.g., 5 minute) interval. This protocol is repeated for each subsequent time interval.

Exemplary algorithms for use in the MPC, or algorithm, modules and microcontrollers are detailed in U.S. Pat. Nos. 8,562,587 and 8,762,070 and U.S. application Ser. Nos. 13/854,963 and 14/154,241 incorporated by reference in their entireties, creating predictive values for controlling the delivery of insulin based on basal rate, meal activities and continuous glucose monitoring. However, other known MPC or PID type delivery system and predictive algorithms may be used.

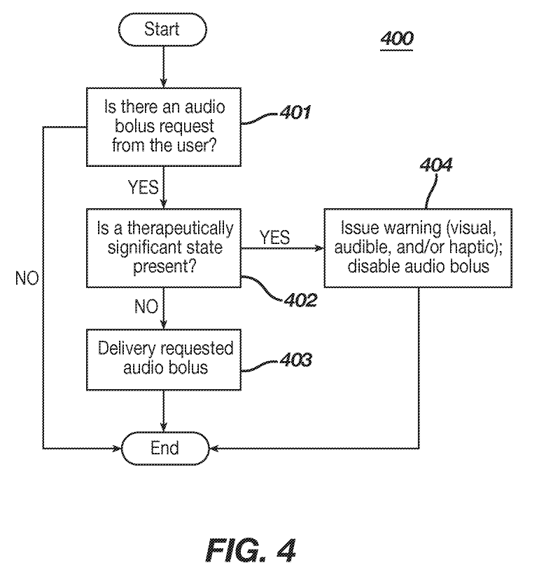

In the system of the invention, and as shown in FIG. 4, a system user inputs an audio bolus request 401 through a device of the system, such as a controller or, and preferably, the drug delivery device. A controller, such as a master microcontroller, MPC module, or like controller within the system then determines whether a therapeutically significant state exists 402 based on one or both of the previous deliveries of insulin above basal rate delivered by the system and bolus deliveries delivered at the user's direction. If no therapeutically significant state exists, the bolus delivery proceeds 403. If it is determined that there is a therapeutically significant state, the controller will not allow the bolus to proceed and the user is provided an audio or visual indication that the audio bolus feature has been disabled 404.

What is claimed is:

1. A drug delivery system, comprising:
a drug delivery device;
at least one controller configured to autonomously modulate drug delivery from the drug delivery device;
a sensor for measuring an analyte of the user;
and an audio bolus feature, wherein the audio bolus feature is automatically disabled if the controller determines a therapeutically significant state is present.

2. The drug delivery system of claim 1, wherein the therapeutically significant state is hypoglycemia or an impending occurrence of hypoglycemia.

3. The drug delivery system of claim 1, wherein the therapeutically significant state is determined by a value for insulin on board.

4. The drug delivery system of claim 3, wherein the audio bolus is disabled if a current IOB value is greater than or equal to a preselected IOB value.
